(19) United States
(12) Patent Application Publication
(10) Pub. No.: US 2023/0242626 A1
(43) Pub. Date: Aug. 3, 2023

(54) COMPOSITION FOR TREATMENT AND PREVENTION OF COVID-19

(71) Applicant: AstraZeneca UK Limited, Cambridge (UK)

(72) Inventors: Albert SCHMELZER, Gaithersburg, MD (US); Sajal PATEL, Gaithersburg, MD (US); Annette MEDINA, Gaithersburg, MD (US); Austin GALLEGOS, Gaithersburg, MD (US)

(21) Appl. No.: 18/052,840

(22) Filed: Nov. 4, 2022

Related U.S. Application Data
(60) Provisional application No. 63/276,410, filed on Nov. 5, 2021.

(51) Int. Cl. C07K 16/10; A61P 31/14

(52) U.S. Cl. CPC C07K 16/10; A61P 31/14; C07K 2317/565

(57) ABSTRACT

The present disclosure provides pharmaceutical compositions comprising antibodies and antigen-binding fragments thereof that specifically bind to the spike protein of SARS-CoV-2 the prevention and treatment of Coronavirus Disease 2019 (COVID-19) in a subject.

### 1. CROSS-REFERENCE TO RELATED APPLICATION

This application claims priority to U.S. Provisional Application No. 63/276,410, filed on Nov. 5, 2021, which is incorporated herein by reference in its entirety.

### 2. REFERENCE TO SEQUENCE LISTING SUBMITTED ELECTRONICALLY

The content of the electronically submitted sequence listing (Name: 2943_2060002_Seqlisting_ST26; Size: 25,601 bytes; and Date of Creation: Nov. 4, 2022) filed with the application is incorporated herein by reference in its entirety.

### 3. FIELD

The present disclosure relates generally to pharmaceutical formulations comprising antibodies or antigen-binding fragments thereof for the prevention and treatment of Coronavirus Disease 2019 (COVID-19) in a subject.

### 4. SUMMARY

Provided herein are pharmaceutical formulations comprising one or more antibodies or antigen-binding fragments thereof that binds to a spike protein of SARS-CoV-2. In some aspects, provided herein are pharmaceutical formulations comprising: (a) a first antibody or antigen-binding fragment thereof that binds to a spike protein of SARS-CoV-2 and optionally a second antibody or antigen-binding fragment thereof that binds to a spike protein of SARS-CoV-2, (b) histidine and/or a pharmaceutically acceptable salt thereof, (c) arginine and/or a pharmaceutically acceptable salt thereof or sucrose, and (d) polysorbate, wherein the formulation has a pH of about 5.5 to 6.5.

In some aspects, the formulation described herein comprises about 15 mM to about 25 mM of (b), optionally wherein the formulation comprises about 20 mM of (b). In some aspects, (b) is histidine/histidine-HCl.

In some aspects, the formulation described herein comprises about 200 to about 250 mM of (c). In some aspects, (c) is arginine and/or a pharmaceutically acceptable salt thereof. In some aspects, (c) is arginine/arginine-HCl. In some aspects, the formulation comprises about 220 mM of (c). In some aspects, (c) is sucrose. In some aspects, the formulation comprises about 240 mM of (c).

In some aspects, the formulation described herein comprises about 0.03% to about 0.05% (w/v) of (d). In some aspects, (d) is polysorbate 80.

In some aspects, the formulation described herein has a pH of about 6.0.

In some aspects, provided herein are pharmaceutical formulations comprising: (a) a first antibody or antigen-binding fragment thereof that binds to a spike protein of SARS-CoV-2 and a second antibody or antigen-binding fragment thereof that binds to a spike protein of SARS-CoV-2, (b) about 20 mM histidine and/or a pharmaceutically acceptable salt thereof, (c) about 220 mM arginine and/or a pharmaceutically acceptable salt thereof, and (d) about 0.04% (w/v) polysorbate 80, wherein the formulation has a pH of about 6.0.

In some aspects, the formulation comprises about 135 mg/mL to about 165 mg/mL of (a). In some aspects, the formulation comprises about 150 mg/mL of (a).

In some aspects, of the pharmaceutical formulations described herein, the formulation comprises about a 1:1 ratio of the first antibody or antigen-binding fragment thereof and the second antibody or antigen-binding fragment thereof.

In some aspects, the formulation is about 2 mL.

In some aspects, the formulation comprises about 300 mg of (a).

In some aspects of the pharmaceutical formulations described herein, the first antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and/or the second antibody or antigen-binding fragment thereof comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects of the pharmaceutical formulations described herein, the first antibody or antigen-binding fragment thereof comprises a variable heavy chain (VH) comprising the amino acid sequence of SEQ ID NO:7 and a variable light chain (VL) comprising the amino acid sequence of SEQ ID NO:8; and/or the second antibody or antigen-binding fragment thereof comprises a variable heavy chain (VH) comprising the amino acid sequence of SEQ ID NO:15 and a variable light chain (VL) comprising the amino acid sequence of SEQ ID NO:16.

In some aspects, the first antibody or antigen-binding fragment thereof is an IgG and/or the second antibody or antigen-binding fragment thereof is an IgG. In some aspects, the first antibody or antigen-binding fragment thereof is an IgG1 and/or the second antibody or antigen-binding fragment thereof is an IgG1. In some aspects, the first antibody or antigen-binding fragment thereof comprises a YTE mutation and/or the second antibody or antigen-binding fragment thereof comprises a YTE mutation.

In some aspects of the pharmaceutical formulations described herein, the first antibody or antigen-binding fragment thereof comprises a heavy chain comprising amino acids 1-460 of SEQ ID NO:17 and a light chain comprising the amino acid sequence of SEQ ID NO:18 and/or wherein the second antibody or antigen-binding fragment thereof comprises a heavy chain comprising amino acids 1-460 of SEQ ID NO:19 and a light chain comprising the amino acid sequence of SEQ ID NO:20.

In some aspects, provided herein are pharmaceutical formulations comprising: (a) an antibody or antigen-binding fragment thereof that binds to a spike protein of SARS-CoV-2, (b) about 20 mM histidine/histidine-HCl, (c) about 240 mM sucrose, and (d) about 0.04% (w/v) polysorbate 80, wherein the formulation has a pH of 6.0.

In some aspects, the formulation comprises about 100 mg/mL of (a). In some aspects, the formulation comprises about 150 mg of (a).

In some aspects, the formulation is about 1.5 mL.

In some aspects of the pharmaceutical formulations described herein, the antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or the antibody or antigen-binding fragment thereof comprises a CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects, the antibody or antigen-binding fragment thereof comprises a variable heavy chain (VH) comprising the amino acid sequence of SEQ ID NO:7 and a variable light chain (VL) comprising the amino acid sequence of SEQ ID NO:8; or the antibody or antigen-binding fragment thereof comprises a variable heavy chain (VH) comprising the amino acid sequence of SEQ ID NO:15 and a variable light chain (VL) comprising the amino acid sequence of SEQ ID NO:16.

In some aspects, the antibody or antigen-binding fragment thereof is an IgG. In some aspects, the antibody or antigen-binding fragment thereof is an IgG1. In some aspects, the antibody or antigen-binding fragment thereof comprises a YTE mutation. In some aspects, the antibody or antigen-binding fragment thereof comprises a TM mutation.

In some aspects, the antibody or antigen-binding fragment thereof comprises a heavy chain comprising amino acids 1-460 of SEQ ID NO:17 and a light chain comprising the amino acid sequence of SEQ ID NO:18; or the antibody or antigen-binding fragment thereof comprises a heavy chain comprising amino acids 1-460 of SEQ ID NO:17 and a light chain comprising the amino acid sequence of SEQ ID NO:18.

In some aspects, of the pharmaceutical formulations are formulated for intramuscular injection. In some aspects, the pharmaceutical formulations are formulated for direct injection into the lateral thigh, gluteal dorsal, or gluteal ventral.

In some aspects of the pharmaceutical formulations described herein, the formulation is stable at 2-8° C. for at least 12 months. In some aspects, the formulation is stable at room temperature for at least 1 week or for at least 2 weeks.

In some aspects, disclosure of vials comprising the pharmaceutical formulation described herein are provided. In some aspects, disclosure of a syringe comprising the pharmaceutical formulation described herein is provided.

In some aspects, disclosed herein are kits comprising a first pharmaceutical formulation and a second pharmaceutical formulation, wherein the first formulation comprises an antibody or antigen-binding fragment thereof comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and the second formulation comprises an antibody or antigen-binding fragment thereof comprising a CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

In some aspects of the kits disclosed herein, the first formulation comprises an antibody or antigen-binding fragment thereof comprising a variable heavy chain (VH) comprising the amino acid sequence of SEQ ID NO:7 and a variable light chain (VL) comprising the amino acid sequence of SEQ ID NO:8; or the second formulation comprises an antibody or antigen-binding fragment thereof comprising a variable heavy chain (VH) comprising the amino acid sequence of SEQ ID NO:15 and a variable light chain (VL) comprising the amino acid sequence of SEQ ID NO:16.

In some aspects of the kits disclosed herein, the first formulation comprises an antibody or antigen-binding fragment thereof comprising a heavy chain comprising amino acids 1-460 of SEQ ID NO:17 and a light chain comprising the amino acid sequence of SEQ ID NO:18; or the second formulation comprises an antibody or antigen-binding fragment thereof comprising a heavy chain comprising amino acids 1-460 of SEQ ID NO:17 and a light chain comprising the amino acid sequence of SEQ ID NO:18.

In some aspects, provided herein are pharmaceutical formulations, vials, syringes, or kits comprising pharmaceutical formulations for use in a method of treating or preventing Coronavirus Disease 2019 (COVID-19) in a subject.

In some aspects, provided herein are methods of treating or preventing Coronavirus Disease 2019 (COVID-19) in a subject comprising administering the pharmaceutical formulations, vials, syringes, or kits described herein to the subject. In some aspects, the pharmaceutical formulations, vials, syringes, kits, or methods described herein prevents or decreases the severity of one or more symptoms of COVID-19. In some aspects, the subject has been exposed to SARS-CoV-2. In some aspects, the subject does not have a known exposure to SARS-CoV-2.

In some aspects, the subject is less than 70 kg. In some aspects, the subject is at least 70 kg and less than 80 kg. In some aspects, the subject is at least 80 kg.

In some aspects, the subject has received an anti-SARS-CoV-2 vaccination. In some aspects, the subject has received at least two anti-SARS-CoV-2 vaccinations against anti-SARS-CoV-2. In some aspects, the subject has not received an anti-SARS-CoV-2 vaccination. In some aspects, the subject has a BMI of 18 to 30 kg/m².

### 5. BRIEF DESCRIPTION OF THE FIGURES

FIG. 1 shows the viscosity of anti-SARS-CoV-2 antibody formulations.

FIG. 2 shows the efficacy of arginine on minimizing viscosity in anti-SARS-CoV-2 antibody formulations.

FIG. 3 shows the NUV CD profiles of anti-SARS-CoV-2 antibody formulations comprising sucrose (buffer 1) or arginine (buffer 2).

FIG. 4 shows the conformational stability of anti-SARS-CoV-2 antibody formulations comprising sucrose (B1) or arginine (B2).

### 6. DETAILED DESCRIPTION

Provided herein are pharmaceutical compositions comprising antibodies (e.g., monoclonal antibodies) or antigen-binding fragments thereof that specifically bind to the spike protein of SARS-CoV-2, e.g., for the treatment and prevention of COVID-19.

### 6.1 Terminology

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-SARS-CoV-2 antibody,” “SARS-CoV-2 antibody” and “antibody that binds to SARS-CoV-2” are used interchangeably herein to refer to an antibody that is capable of binding to SARS-CoV-2. The extent of binding of a SARS-CoV-2 antibody to an unrelated, non-SARS-CoV-2 spike protein can be less than about 10% of the binding of the antibody to SARS-CoV-2 as measured, e.g., using ForteBio or Biacore. In some aspects provided herein, a SARS-CoV-2 antibody is also capable of binding to SARS-1. In some aspects provided herein, a SARS-CoV-2 antibody does not bind to SARS-1.

The terms “anti-spike protein of SARS-CoV-2 antibody,” “SARS-CoV-2 spike protein antibody” and “antibody that binds to the spike protein of SARS-CoV-2” are used interchangeably herein to refer to an antibody that is capable of binding to the spike protein of SARS-CoV-2 with sufficient affinity such that the antibody is useful as a diagnostic and/or therapeutic agent in targeting SARS-CoV-2. The extent of binding of a SARS-CoV-2 spike protein antibody to an unrelated, non-SARS-CoV-2 spike protein can be less than about 10% of the binding of the antibody to SARS-CoV-2 spike protein as measured, e.g., using ForteBio or Biacore. In some aspects provided herein, a SARS-CoV-2 spike protein antibody is also capable of binding to the spike protein of SARS-1. In some aspects provided herein, a SARS-CoV-2 spike protein antibody does not bind to the spike protein of SARS-1.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The term “complementarity determining region” or “CDR” as used herein refers to each of the regions of an antibody variable domain which are hypervariable in sequence and/or form structurally defined loops (hypervariable loops) and/or contain the antigen-contacting residues. Antibodies can comprise six CDRs, e.g., three in the VH and three in the VL.

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-134 |
| L2 | L50-L56 | L50-L56 | L50-L56 |
| L3 | L89-197 | L89-L97 | L89-197 |
| H1 | H31-H3SB | H26-H35B | H26-H32..34 |
| | | (Kabat Numbering) | |
| H1 | H31-H35 | H26-H35 | H26-H32 |
| | | (Chothia Numbering) | |
| H2 | H50-H65 | H50-H58 | H52-H56 |
| H3 | H95-H102 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some aspects, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen-binding domain and the epitope. Accordingly, in some aspects, an antibody that “specifically binds” to the spike protein of SARS-CoV-2 can also bind to the spike protein of one or more related viruses (e.g., SARS-1) and/or can also bind to variants of the spike protein of SARS-CoV-2, but the extent of binding to an unrelated, non-SARS-CoV-2 spike protein is less than about 10% of the binding of the antibody to the spike protein of SARS-CoV—as measured, e.g., using ForteBio or Biacore.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that may be used to enable delivery of a drug, e.g., a combination of antibodies or antigen-binding fragments thereof that specifically bind to the spike protein of SARS-CoV-2 to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

The term “therapeutically effective amount” refers to an amount of a drug, e.g., a combination of antibodies or antigen-binding fragments thereof effective to treat a disease or disorder in a subject.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already diagnosed with or suspected of having the disorder. Patients or subjects in need of treatment can include those diagnosed with coronavirus 2019 (COVID-19) and those who have been infected with severe acute respiratory syndrome coronavirus 2 (SARS-CoV-2).

As used herein, the term “COVID-19” refers to an infection with SARS-CoV-2. A subject with COVID-19 can be symptomatic or asymptomatic.

As used herein, a subject who has “received an anti-SARS-CoV-2 vaccination” refers to a subject who has received at least one dose or an anti-SARS-CoV-2 vaccine. The vaccine can be, for example, a messenger RNA (mRNA) vaccine or a DNA vaccine. As used herein a subject who has “received at least two anti-SARS-CoV-2 vaccinations” refers to a subject who has received at least two doses of an anti-SARS-CoV-2 vaccine. The two doses can be of the same vaccine or can be of different vaccines.

Alternatively, the pharmacologic and/or physiologic effect may be prophylactic, i.e., the effect completely or partially prevents a disease or symptom thereof. In this respect, the disclosed method comprises administering a “prophylactically effective amount” of a drug (e.g., a combination of antibodies or antigen-binding fragments thereof). A “prophylactically effective amount” refers to an amount effective, at dosages and for periods of time necessary, to achieve a desired prophylactic result (e.g., prevention of COVID-19 or SARS-CoV-2 infection).

As used herein, the terms “combination” and “administered in combination” refer to the administration of one antibody or antigen-binding fragment thereof with another antibody or antigen-binding fragment thereof. The antibodies or antigen-binding fragments thereof in the combination can be administered simultaneously or sequentially. The antibodies or antigen-binding fragments thereof in the combination can be administered in the same or in different compositions.

As provided herein, reference to a “first” antibody or antigen-binding fragment thereof and a “second” antibody or antigen-binding fragment in a combination do not refer to the order of administration. The “first antibody or antigen-binding fragment thereof,” can be administered either before or after the “second antibody or antigen-binding fragment thereof.”

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

Any compositions or methods provided herein can be combined with one or more of any of the other compositions and methods provided herein.

### 6.2 Compositions Comprising Anti-SARS-CoV-2 Antibodies or Antigen-Binding Fragments Thereof

Provided herein are compositions comprising anti-SARS-CoV-2 antibodies or antigen-binding fragments thereof for use in methods of treating or preventing COVID-19 (i.e., a SARS-CoV-2 infection) in a subject. In some aspects, the methods comprise administering a first and second anti-SARS-CoV-2 antibody or antigen-binding fragment thereof in one or more pharmaceutical formulations described herein.

As provided herein, a pharmaceutical formulation can comprise at least one antibody or antigen-binding fragment thereof that binds to a spike protein of SARS-CoV-2. In some aspects, a pharmaceutical formulation comprises no more than one antibody or antigen-binding fragment thereof that binds to a spike protein of SARS-CoV-2. In some aspects, a pharmaceutical formulation comprises two antibodies or antigen-binding fragments thereof, wherein each antibody or fragment binds to a spike protein of SARS-CoV-2.

As provided herein, a pharmaceutical formulation comprising at least one antibody or antigen-binding fragment thereof that binds to a spike protein of SARS-CoV-2 can further comprise histidine and/or a pharmaceutically acceptable salt thereof. In some aspects, the pharmaceutically acceptable salt is HCl. Accordingly, a pharmaceutical formulation can comprise histidine/histidine-HCl. In some aspects, a pharmaceutical formulation comprises about 15 nM to about 25 mM of histidine and/or a pharmaceutically acceptable salt thereof (e.g., about 15 nM to about 25 mM histidine/histidine-HCl). In some aspects, a pharmaceutical formulation comprises about 15 nM to about 20 mM of histidine and/or a pharmaceutically acceptable salt thereof (e.g., about 15 nM to about 20 mM histidine/histidine-HCl). In some aspects, a pharmaceutical formulation comprises about 20 nM to about 25 mM of histidine and/or a pharmaceutically acceptable salt thereof (e.g., about 20 nM to about 25 mM histidine/histidine-HCl). In some aspects, a pharmaceutical formulation comprises about 18 nM to about 22 mM of histidine and/or a pharmaceutically acceptable salt thereof (e.g., about 18 nM to about 22 mM histidine/histidine-HCl). In some aspects, a pharmaceutical formulation comprises about 20 mM of histidine and/or a pharmaceutically acceptable salt thereof (e.g., about 20 mM histidine/histidine-HCl).

As provided herein, a pharmaceutical formulation comprising at least one antibody or antigen-binding fragment thereof that binds to a spike protein of SARS-CoV-2 can further comprise arginine and/or a pharmaceutically acceptable salt thereof. In some aspects, the pharmaceutically acceptable salt is HCl. Accordingly, a pharmaceutical formulation can comprise arginine/arginine-HCl. In some aspects, a pharmaceutical formulation comprises about 200 nM to about 250 mM of arginine and/or a pharmaceutically acceptable salt thereof (e.g., about 200 nM to about 250 mM arginine/arginine-HCl). In some aspects, a pharmaceutical formulation comprises about 210 nM to about 230 mM of arginine and/or a pharmaceutically acceptable salt thereof (e.g., about 210 nM to about 230 mM arginine/arginine-HCl). In some aspects, a pharmaceutical formulation comprises about 220 mM of arginine and/or a pharmaceutically acceptable salt thereof (e.g., about 220 mM arginine/arginine-HCl).

As provided herein, a pharmaceutical formulation comprising at least one antibody or antigen-binding fragment thereof that binds to a spike protein of SARS-CoV-2 can further comprise sucrose. In some aspects, a pharmaceutical formulation comprises about 200 nM to about 250 mM of sucrose. In some aspects, a pharmaceutical formulation comprises about 230 nM to about 250 mM of sucrose. In some aspects, a pharmaceutical formulation comprises about 240 mM of sucrose.

As provided herein, a pharmaceutical formulation comprising at least one antibody or antigen-binding fragment thereof that binds to a spike protein of SARS-CoV-2 can further comprise polysorbate. In some aspects, a pharmaceutical formulation comprises polysorbate 80. In some aspects, a pharmaceutical formulation comprises about 0.03% to about 0.05% (w/v) of polysorbate (e.g., about 0.03% to about 0.05% (w/v) polysorbate 80). In some aspects, a pharmaceutical formulation comprises about 0.04% (w/v) of polysorbate (e.g., about 0.04% (w/v) polysorbate 80).

A pharmaceutical composition provided herein can have a pH of about 5.5 to about 6.5. In some aspects, a pharmaceutical composition provided herein has a pH of about 5.8 to about 6.2. In some aspects, a pharmaceutical composition provided herein has a pH of about 5.5 to about 6.0. In some aspects, a pharmaceutical composition provided herein has a pH of about 5.8 to about 6.0. In some aspects, a pharmaceutical composition provided herein has a pH of about 6.0 to about 6.5. In some aspects, a pharmaceutical composition provided herein has a pH of about 6.0 to about 6.2.

In some aspects provided herein, a pharmaceutical formulation comprising at least one antibody or antigen-binding fragment thereof that binds to a spike protein of SARS-CoV-2 further comprises histidine and/or a pharmaceutically acceptable salt thereof, arginine and/or a pharmaceutically acceptable salt thereof, and polysorbate. In some aspects, such a pharmaceutical formulation has a pH of about 5.5 to about 6.5. In some aspects, the pH is about 6.0.

In some aspects provided herein, a pharmaceutical formulation comprising at least one antibody or antigen-binding fragment thereof that binds to a spike protein of SARS-CoV-2 further comprises about 15 mM to about 25 mM histidine and/or a pharmaceutically acceptable salt thereof (e.g., histidine/histidine-HCl), about 220 mM arginine and/or a pharmaceutically acceptable salt thereof (e.g., arginine/arginine-HCl), and about 0.03% to about 0.05% (w/v) polysorbate (e.g., polysorbate 80). In some aspects, such a pharmaceutical formulation has a pH of about 5.5 to about 6.5. In some aspects, the pH is about 6.0.

In some aspects provided herein, a pharmaceutical formulation comprising at least one antibody or antigen-binding fragment thereof that binds to a spike protein of SARS-CoV-2 further comprises about 20 mM histidine and/or a pharmaceutically acceptable salt thereof (e.g., histidine/histidine-HCl), about 220 mM arginine and/or a pharmaceutically acceptable salt thereof (e.g., arginine/arginine-HCl), and about 0.04% (w/v) polysorbate (e.g., polysorbate 80). In some aspects, such a pharmaceutical formulation has a pH of about 5.5 to about 6.5. In some aspects, the pH is about 6.0.

In some aspects provided herein, a pharmaceutical formulation comprising at least one antibody or antigen-binding fragment thereof that binds to a spike protein of SARS-CoV-2 further comprises histidine and/or a pharmaceutically acceptable salt thereof, sucrose, and polysorbate. In some aspects, such a pharmaceutical formulation has a pH of about 5.5 to about 6.5. In some aspects, the pH is about 6.0.

In some aspects provided herein, a pharmaceutical formulation comprises about 135 mg/mL to about 165 mg/mL of at least one antibody or antigen-binding fragment thereof that binds to a spike protein of SARS-CoV-2. In some aspects provided herein, a pharmaceutical formulation comprises about 135 mg/mL to about 165 mg/mL of a mixture of two antibodies or antigen-binding fragments thereof that bind to a spike protein of SARS-CoV-2. The mixture can comprise about a 1:1 ratio of a first antibody or antigen-binding fragment thereof and a second antibody or antigen-binding fragment thereof.

In some aspects provided herein, a pharmaceutical formulation comprises about 150 mg/mL of at least one antibody or antigen-binding fragment thereof that binds to a spike protein of SARS-CoV-2. In some aspects provided herein, a pharmaceutical formulation comprises about 150 mg/mL of a mixture of two antibodies or antigen-binding fragments thereof that bind to a spike protein of SARS-CoV-2. The mixture can comprise about a 1:1 ratio of a first antibody or antigen-binding fragment thereof and a second antibody or antigen-binding fragment thereof.

In some aspects provided herein, a pharmaceutical formulation is about 1.5 mL. In some aspects provided herein, a pharmaceutical formulation is about 2 mL.

In some aspects provided herein, a pharmaceutical formulation comprises about 150 mg of a mixture of an antibody or antigen-binding fragment thereof that bind to a spike protein of SARS-CoV-2. In some aspects provided herein, a pharmaceutical formulation comprises about 300 mg of a mixture of two antibodies or antigen-binding fragments thereof that bind to a spike protein of SARS-CoV-2.

In some aspects provided herein, a pharmaceutical formulation is formulated for intramuscular injection. The intramuscular injection can be into the lateral thigh, gluteal dorsal, or gluteal ventral.

Also provided herein are vials and syringes comprising a pharmaceutical formulation provided herein.

### 6.3 Antibodies and Antigen-Binding Fragments Thereof

In some aspects, provided herein are pharmaceutical formulations comprising antibodies (e.g., monoclonal antibodies, such as human antibodies) or antigen-binding fragments thereof that bind to the spike protein of SARS-CoV-2. The amino acid sequence of the spike protein of SARS-CoV-2 is provided in SEQ ID NO:22:

(SEQ ID NO: 22)
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

Amino acids 1-12 of SEQ ID NO:22 are the signal peptide of the spike protein. Therefore, the mature version of the spike protein of SARS-CoV-2 contains amino acids 13-1273 of SEQ ID NO:22. Amino acids 13-1213 of SEQ ID NO:22 correspond to the extracellular domain; amino acids 1214-1234 correspond to the transmembrane domain; and amino acids 1235-1273 correspond to the cytoplasmic domain.

In some aspects, an antibody or antigen-binding fragment thereof for use in a pharmaceutical formulation described herein, i.e., a first antibody or antigen-binding fragment thereof and/or a second antibody or antigen-binding fragment thereof, binds to the spike protein of SARS-CoV-2 and specifically binds to the receptor binding domain (RBD) of the spike protein of SARS-CoV-2.

In some aspects, the first antibody or antigen-binding fragment thereof for use in a pharmaceutical formulation described herein and the second antibody or antigen-binding fragment thereof described herein each bind to distinct, non-overlapping epitopes on the RBD of the spike protein of SARS-CoV-2.

In some aspects, the first antibody or antigen-binding fragment thereof for use in a pharmaceutical formulation described herein is antibody clone 2196. In some aspects, the second antibody or antigen-binding fragment thereof is antibody clone 2130.

In some aspects, an antibody or antigen-binding fragment thereof for use in a pharmaceutical formulation described herein, that specifically binds to the spike protein of SARS-CoV-2 cross-reacts with SARS-CoV. In some aspects, an antibody or antigen-binding fragment thereof for use in a pharmaceutical formulation described herein, that specifically binds to the spike protein of SARS-CoV-2 does not cross-react with SARS-CoV.

In some aspects, an antibody or antigen-binding fragment thereof for use in a pharmaceutical formulation described herein binds to the spike protein of SARS-CoV-2 and comprises the six CDRs of an antibody listed in Table 1 (i.e., the three VH CDRs of the antibody and the three VL CDRs of the same antibody).

TABLE 1
Antibody Sequences

| | SEQ | | | | |
|---|---|---|---|---|---|
| | ID | | CDR1 | CDR2 | CDR3 |
| Clone | NO | Sequence (Description) | (SEQ ID NO) | (SEQ ID NO) | (SEQ ID NO) |
| 2196 | 7 | QMQLVQSGPEVKKPGTSVKVSCKASGFTFMSSAVQW | GFTFMSSA | IVIGSGNT | AAPYCSSISC |
| | | VRQARGQRLEWIGWIVIGSGNTNYAQKFQERVTITRD | (SEQ ID NO: | (SEQ ID NO: | NDGFDI |
| | | MSTSTAYMELSSLRSEDTAVYYCAAPYCSSISCNDGF | 1) | 2) | (SEQ ID NO: |
| | | DIWGQGTMVTVSS (Heavy chain variable region) | | | 3) |
| 2196 | 8 | EIVLTQSPGTLSLSPGERATLSCRASQSVSSSYLAWYQ | QSVSSSY | GAS | QHYGSSRG |
| | | QKPGQAPRLLIYGASSRATGIPDRFSGSGSGTDFTLTIS | (SEQ ID NO: | (SEQ ID NO: | WT |
| | | RLEPEDFAVYYCQHYGSSRGWTFGQGTKVEIK (Light | 4) | 5) | (SEQ ID NO: |
| | | chain variable region) | | | 6) |
| 2196 | 17 | QMQLVQSGPEVKKPGTSVKVSCKASGFTFMSSAVQW | | | |
| | | VRQARGQRLEWIGWIVIGSGNTNYAQKFQERVTITRD | | | |
| | | MSTSTAYMELSSLRSEDTAVYYCAAPYCSSISCNDGF | | | |
| | | DIWGQGTMVTVSSASTKGPSVFPLAPSSKSTSGGTAA | | | |
| | | LGCLVKDYFPEPVTVSWNSGALTSGVHTFPAVLQSSG | | | |
| | | LYSLSSVVTVPSSSLGTQTYICNVNHKPSNTKVDKRVE | | | |
| | | PKSCDKTHTCPPCPAPEFEGGPSVFLFPPKPKDTLYITR | | | |
| | | EPEVTCVVVDVSHEDPEVKFNWYVDGVEVHNAKTKP | | | |
| | | REEQYNSTYRVVSVLTVLHQDWLNGKEYKCKVSNK | | | |
| | | ALPASIEKTISKAKGQPREPQVYTLPPSREEMTKNQVS | | | |
| | | LTCLVKGFYPSDIAVEWESNGQPENNYKTTPPVLDSD | | | |
| | | GSFFLYSKLTVDKSRWQQGNVFSCSVMHEALHNHYT | | | |
| | | QKSLSLSPGK (Full length heavy chain*; | | | |
| | | YTE underlined; TM bold and underlined) | | | |
| 2196 | 18 | EIVLTQSPGTLSLSPGERATLSCRASQSVSSSYLAWYQ | | | |
| | | QKPGQAPRLLIYGASSRATGIPDRFSGSGSGTDFTLTIS | | | |
| | | RLEPEDFAVYYCQHYGSSRGWTFGQGTKVEIKRTVA | | | |
| | | APSVFIFPPSDEQLKSGTASVVCLLNNFYPREAKVQW | | | |
| | | KVDNALQSGNSQESVTEQDSKDSTYSLSSTLTLSKAD | | | |
| | | YEKHKVYACEVTHQGLSSPVTKSFNRGEC (Full length | | | |
| | | light chain) | | | |
| 2130 | 15 | EVQLVESGGGLVKPGGSLRLSCAASGFTFRDVWMSW | GFTFRDVW | IKSKIDGGTT | TTAGSYYYD |
| | | VRQAPGKGLEWVGRIKSKIDGGTTDYAAPVKGRFTIS | (SEQ ID | (SEQ ID | TVGPGLPEG |
| | | RDDSKNTLYLQMNSLKTEDTAVYYCTTAGSYYYDTV | NO: 9) | NO: 10) | KFDY (SEQ |
| | | GPGLPEGKFDYWGQGTLVTVSS (Heavy chain | | | ID NO: 11) |
| | | variable region) | | | |
| 2130 | 16 | DIVMTQSPDSLAVSLGERATINCKSSQSVLYSSNNKNY | QSVLYSSN | WAS (SEQ ID | QQYYSTLT |
| | | LAWYQQKPGQPPKLLMYWASTRESGVPDRFSGSGSG | NKNY (SEQ | NO:13) | (SEQ ID |
| | | AEFTLTISSLQAEDVAIYYCQQYYSTLTFGGGTKVEIK | ID NO: 12) | | NO: 14) |
| | | (Light chain variable region) | | | |
| 2130 | 19 | EVQLVESGGGLVKPGGSLRLSCAASGFTFRDVWMSW | | | |
| | | VRQAPGKGLEWVGRIKSKIDGGTTDYAAPVKGRFTIS | | | |
| | | RDDSKNTLYLQMNSLKTEDTAVYYCTTAGSYYYDTV | | | |
| | | GPGLPEGKFDYWGQGTLVTVSSASTKGPSVFPLAPSS | | | |
| | | KSTSGGTAALGCLVKDYFPEPVTVSWNSGALTSGVHT | | | |
| | | FPAVLQSSGLYSLSSVVTVPSSSLGTQTYICNVNHKPS | | | |
| | | NTKVDKRVEPKSCDKTHTCPPCPAPEFEGGPSVFLFPP | | | |
| | | KPKDTLYITREPEVTCVVVDVSHEDPEVKFNWYVDG | | | |
| | | VEVHNAKTKPREEQYNSTYRVVSVLTVLHQDWLNG | | | |
| | | KEYKCKVSNKALPASIEKTISKAKGQPREPQVYTLPPS | | | |
| | | REEMTKNQVSLTCLVKGFYPSDIAVEWESNGQPENN | | | |
| | | YKTTPPVLDSDGSFFLYSKLTVDKSRWQQGNVFSCSV | | | |
| | | MHEALHNHYTQKSLSLSPGK (Full length heavy | | | |
| | | chain*; YTE underlined; TM bold and under- | | | |
| | | lined) | | | |
| 2130 | 20 | DIVMTQSPDSLAVSLGERATINCKSSQSVLYSSNNKNY | | | |
| | | LAWYQQKPGQPPKLLMYWASTRESGVPDRFSGSGSG | | | |
| | | AEFTLTISSLQAEDVAIYYCQQYYSTLTFGGGTKVEIK | | | |
| | | RTVAAPSVFIFPPSDEQLKSGTASVVCLLNNFYPREAK | | | |
| | | VQWKVDNALQSGNSQESVTEQDSKDSTYSLSSTLTLS | | | |
| | | KADYEKHKVYACEVTHQGLSSPVTKSFNRGEC (Full | | | |
| | | length light chain) | | | |

*The full length leavy chain sequences provided in Table 1 contain a terminal lysine which can be post-translationally clipped, so that the predominant form of the heavy chains can be amino acids 1-460 of SEQ ID NO: 19 and amino acids 1-460 of SEQ ID NO: 17.

In some aspects, the first antibody or antigen-binding fragment thereof for use in a pharmaceutical formulation described herein and the second antibody or antigen-binding fragment thereof described herein each bind to the spike protein of SARS-CoV-2 and comprise the two VH and two VL of the antibodies listed in Table 1.

In some aspects, the antibodies or antigen-binding fragments thereof for use in a pharmaceutical formulation described herein may be described by its 3 VL CDRs and/or or its 3 VH CDRs.

In some aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, provided herein are pharmaceutical formulations comprising antibodies or antigen-binding fragments thereof that specifically bind to the spike protein of SARS-CoV-2 and comprise the Chothia VH and VL CDRs of the antibodies listed in Table 1. In some aspects, antibodies or antigen-binding fragments thereof that specifically bind to the spike protein of SARS-CoV-2 comprise one or more CDRs, in which the Chothia and Kabat CDRs have the same amino acid sequence. In some aspects, provided herein are antibodies and antigen-binding fragments thereof that specifically bind to the spike protein of SARS-CoV-2 and comprise combinations of Kabat CDRs and Chothia CDRs.

In some aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In some aspects, provided herein are antibodies and antigen-binding fragments thereof that specifically bind to the spike protein of SARS-CoV-2 and comprise the IMGT VH and VL CDRs of an antibody listed in Table 1, for example, as described in Lefranc M-P (1999) supra and Lefranc M-P et al., (1999) supra).

In some aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dithel, eds., Chapter 31, pp. 422-439, Springer-Verlag, Berlin (2001). In some aspects, provided herein are antibodies or antigen-binding fragments thereof that specifically bind to the spike protein of SARS-CoV-2 and comprise VH and VL CDRs of an antibody listed in Table 1 as determined by the method in MacCallum R M et al.

In some aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In some aspects, provided herein are antibodies or antigen-binding fragments thereof that specifically bind to the spike protein of SARS-CoV-2 and comprise VH and VL CDRs of an antibody listed in Table 1 as determined by the AbM numbering scheme.

In some aspects, provided herein are antibodies that comprise a heavy chain and a light chain. Non-limiting examples of human constant region sequences have been described in the art, e.g., see U.S. Pat. No. 5,693,780 and Kabat E A et al., (1991) supra.

With respect to the heavy chain, in some aspects, the heavy chain of an antibody described herein can be an alpha (α), delta (δ), epsilon (ε), gamma (γ) or mu (μ) heavy chain. In some aspects, the heavy chain of an antibody described can comprise a human alpha (α), delta (δ), epsilon (ε), gamma (γ) or mu (μ) heavy chain. In some aspects, an antibody described herein, which immunospecifically binds to the spike protein of SARS-CoV-2, comprises a heavy chain wherein the amino acid sequence of the VH domain comprises an amino acid sequence set forth in Table 1 and wherein the constant region of the heavy chain comprises the amino acid sequence of a human gamma (γ) heavy chain constant region (e.g., a human IgG1 heavy chain constant region). In some aspects, an antibody described herein, which specifically binds to the spike protein of SARS-CoV-2, comprises a heavy chain wherein the amino acid sequence of the VH domain comprises a sequence set forth in Table 1, and wherein the constant region of the heavy chain comprises the amino acid of a human heavy chain described herein or known in the art.

In some aspects, the light chain of an antibody or antigen-binding fragment thereof described herein is a human kappa light chain or a human lambda light chain. In some aspects, an antibody described herein, which immunospecifically binds to the spike protein of SARS-CoV-2 comprises a light chain wherein the amino acid sequence of the VL domain comprises a sequence set forth in Table 1 and wherein the constant region of the light chain comprises the amino acid sequence of a human kappa or lambda light chain constant region.

In some aspects, the antibodies or antigen-binding fragments thereof described herein, which immunospecifically bind to the spike protein of SARS-CoV-2 comprise a light chain wherein the amino acid sequence of the VL domain comprises a sequence set forth in Table 1, and wherein the constant region of the light chain comprises the amino acid sequence of a human kappa light chain constant region.

In some aspects, the light chain of an antibody described herein is a lambda light chain. In some aspects, an antibody described herein, which immunospecifically binds to the spike protein of SARS-CoV-2 comprises a light chain wherein the amino acid sequence of the VL domain comprises a sequence set forth in Table 1 and wherein the constant region of the light chain comprises the amino acid sequence of a human lambda light chain constant region.

In some aspects, the antibodies or antigen-binding fragments thereof for use in a pharmaceutical formulation described herein, which immunospecifically bind to the spike protein of SARS-CoV-2 comprise a VH domain and a VL domain comprising any amino acid sequence described herein, and wherein the constant regions comprise the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody for use in a pharmaceutical formulation described herein, which immunospecifically binds to the spike protein of SARS-CoV-2 comprises a VH domain and a VL domain comprising any amino acid sequence described herein, and wherein the constant regions comprise the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Fc region engineering is used in the art, e.g., to extend the half-life of therapeutic antibodies and antigen-binding fragments thereof and protect from degradation in vivo. In some aspects, the Fc region of an IgG antibody or antigen-binding fragment can be modified in order to increase the affinity of the IgG molecule for the Fc Receptor-neonate (FcRn), which mediates IgG catabolism and protects IgG molecules from degradation. Suitable Fc region amino acid substitutions or modifications are known in the art and include, for example, the triple substitution M252Y/S254T/T256E (referred to as “YTE”) (see, e.g., U.S. Pat. No. 7,658,921; U.S. Patent Application Publication 2014/0302058; and Yu et al., Antimicrob. Agents Chemother., 61(1): e01020-16 (2017)). In some aspects, an antibody or antigen-binding binding fragment (e.g., monoclonal antibody or fragment) that binds to the spike protein of SARS-CoV-2 comprises an Fc region comprising the YTE mutation.

The triple mutation (TM) L234F/L235E/P331S (according to European Union numbering convention; Sazinsky et al. Proc Natl Acad Sci USA, 105:20167-20172 (2008)) in the heavy chain constant region can significantly reduce IgG effector function. In some aspects, an IgG1 sequence comprising the triple mutation comprises the of SEQ ID NO:21.

(SEQ ID NO: 21)
EPKSSDKTHTCPPCPAPEFEGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPASIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some aspects, one, two, or more mutations (e.g., amino acid substitutions) are introduced into the Fc region of an antibody or antigen-binding fragment thereof for use in a pharmaceutical formulation described herein (e.g., into the CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity.

In some aspects, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some aspects, one, two, or more mutations (e.g., amino acid substitutions) are introduced into the Fc region of an antibody or antigen-binding fragment thereof for use in a pharmaceutical formulation described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody or antigen-binding fragment thereof for an Fc receptor (e.g., an activated Fc receptor) on the surface of an effector cell. Mutations in the Fc region that decrease or increase affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some aspects, one, two, or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody or antigen-binding fragment thereof in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody or antigen-binding fragment thereof in vivo. In some aspects, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the antibody or antigen-binding fragment thereof in vivo. In some aspects, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody or antigen-binding fragment thereof in vivo. In some aspects, the antibodies or antigen-binding fragments thereof may have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some aspects, the constant region of the IgG1 comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some aspects, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In some aspects, one, two, or more amino acid substitutions are introduced into an IgG constant domain Fc region to alter the effector function(s) of the antibody or antigen-binding fragment thereof. For example, one or more amino acids selected from amino acid residues 234, 235, 236, 237, 297, 318, 320 and 322, numbered according to the EU index as in Kabat, can be replaced with a different amino acid residue such that the antibody or antigen-binding fragment thereof has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some aspects, the deletion or inactivation (through point mutations or other means) of a constant region domain may reduce Fc receptor binding of the circulating antibody or antigen-binding fragment thereof thereby increasing tumor localization. See, e.g., U.S. Pat. Nos. 5,585,097 and 8,591,886 for a description of mutations that delete or inactivate the constant domain and thereby increase tumor localization. In some aspects, one or more amino acid substitutions can be introduced into the Fc region to remove potential glycosylation sites on Fc region, which may reduce Fc receptor binding (see, e.g., Shields R L et al., (2001) J Biol Chem 276: 6591-604).

In some aspects, one or more amino acids selected from amino acid residues 322, 329, and 331 in the constant region, numbered according to the EU index as in Kabat, can be replaced with a different amino acid residue such that the antibody or antigen-binding fragment thereof has altered C1q binding and/or reduced or abolished complement dependent cytotoxicity (CDC). This approach is described in further detail in U.S. Pat. No. 6,194,551 (Idusogie et al). In some aspects, one or more amino acid residues within amino acid positions 231 to 238 in the N-terminal region of the CH2 domain are altered to thereby alter the ability of the antibody to fix complement. This approach is described further in International Publication No. WO 94/29351. In some aspects, the Fc region is modified to increase the ability of the antibody or antigen-binding fragment thereof to mediate antibody dependent cellular cytotoxicity (ADCC) and/or to increase the affinity of the antibody or antigen-binding fragment thereof for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU index as in Kabat. This approach is described further in International Publication No. WO 00/42072.

In some aspects, the antibodies or antigen-binding fragments thereof described herein comprise the constant domain of an IgG1 with a mutation (e.g., substitution) at position 267, 328, or a combination thereof, numbered according to the EU index as in Kabat. In some aspects, an antibody or antigen-binding fragment thereof for use in a pharmaceutical formulation described herein comprises the constant domain of an IgG1 with a mutation (e.g., substitution) selected from the group consisting of S267E, L328F, and a combination thereof. In some aspects, an antibody or antigen-binding fragment thereof for use in a pharmaceutical formulation described herein comprises the constant domain of an IgG1 with a S267E/L328F mutation (e.g., substitution). In some aspects, an antibody or antigen-binding fragment thereof for use in a pharmaceutical formulation described herein comprising the constant domain of an IgG1 with a S267E/L328F mutation (e.g., substitution) has an increased binding affinity for FcγRIIA, FcγRIIB, or FcγRIIA and FcγRIIB.

Engineered glycoforms may be useful for a variety of purposes, including but not limited to enhancing or reducing effector function. Methods for generating engineered glycoforms in an antibody or antigen-binding fragment thereof for use in a pharmaceutical formulation described herein include but are not limited to those disclosed, e.g., in Umaña P et al., (1999) Nat Biotechnol 17: 176-180; Davies J et al., (2001) Biotechnol Bioeng 74: 288-294; Shields R L et al., (2002) J Biol Chem 277: 26733-26740; Shinkawa T et al., (2003) J Biol Chem 278: 3466-3473; Niwa R et al., (2004) Clin Cancer Res 1: 6248-6255; Presta L G et al., (2002) Biochem Soc Trans 30: 487-490; Kanda Y et al., (2007) Glycobiology 17: 104-118; U.S. Pat. Nos. 6,602,684; 6,946,292; and 7,214,775; U.S. Patent Publication Nos. US 2007/0248600; 2007/0178551; 2008/0060092; and 2006/0253928; International Publication Nos. WO 00/61739; WO 01/292246; WO 02/311140; and WO 02/30954; Potillegent™ technology (Biowa, Inc. Princeton, N.J.); and GlycoMAb® glycosylation engineering technology (Glycart biotechnology AG, Zurich, Switzerland). See also, e.g., Ferrara C et al., (2006) Biotechnol Bioeng 93: 851-861; International Publication Nos. WO 07/039818; WO 12/130831; WO 99/054342; WO 03/011878; and WO 04/065540.

In some aspects, any of the constant region mutations or modifications described herein can be introduced into one or both heavy chain constant regions of an antibody or antigen-binding fragment thereof for use in a pharmaceutical formulation described herein having two heavy chain constant regions.

In some aspects, the first antibody or antigen-binding fragment thereof and the second antibody or antigen-binding fragment thereof each inhibit binding of SARS-CoV-2 to angiotensin converting enzyme 2 (ACE2).

In some aspects, the first antibody or antigen-binding fragment thereof and the second antibody or antigen-binding fragment thereof each neutralize SARS-CoV-2.

In some aspects, the first and second antigen-binding fragments disclosed herein comprise a Fab, Fab′, F(ab′)2, single chain Fv (scFv), disulfide linked Fv, V-NAR domain, IgNar, IgGΔCH2, minibody, F(ab′)3, tetrabody, triabody, diabody, single-domain antibody, (scFv)2, or scFv-Fc.

In some aspects, an antigen-binding fragment as described herein that specifically binds to the spike protein of SARS-CoV-2, is selected from the group consisting of a Fab, Fab′, F(ab′)2, and scFv, wherein the Fab, Fab′, F(ab′)2, or scFv comprises a heavy chain variable region sequence and a light chain variable region sequence of an antibody or antigen-binding fragment thereof that specifically binds to the spike protein of SARS-CoV-2 or to SARS-CoV-2. A Fab, Fab′, F(ab′)2, or scFv can be produced by any technique known to those of skill in the art. In some aspects, the Fab, Fab′, F(ab′)2, or scFv further comprises a moiety that extends the half-life of the antibody in vivo. The moiety is also termed a “half-life extending moiety.” Any moiety known to those of skill in the art for extending the half-life of a Fab, Fab′, F(ab′)2, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, the Fab, Fab′, F(ab′)2, or scFv can be modified by the addition of one or more C-terminal amino acids for attachment of the half-life extending moiety. In some aspects the half-life extending moiety is polyethylene glycol or human serum albumin. In some aspects, the Fab, Fab′, F(ab′)2, or scFv is fused to a Fc region.

An antibody or antigen-binding fragment thereof that binds to the spike protein of SARS-CoV-2 can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I) carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies or antigen-binding fragments thereof can be used to detect the spike protein of SARS-CoV-2 or to SARS-CoV-2.

The following examples are offered by way of illustration and not by way of limitation.

### 7. EXAMPLES

The 2196 antibody used throughout the Examples section (and in the corresponding Figures) comprises a heavy chain comprising amino acids 1-460 of SEQ ID NO:17 and a light chain comprising the amino acid sequence of SEQ ID NO:18. The 2130 antibody used throughout the Examples section (and in the corresponding Figures) comprises a heavy chain comprising amino acids 1-460 of SEQ ID NO:19 and a light chain comprising the amino acid sequence of SEQ ID NO:20.

### Example 1: Formulation of Anti-SARS-CoV-2 Antibodies

The 2196+2130 antibodies were selected for use in a combination therapy (referred to as “2196+2130” herein). The 2196 and the 2130 antibodies that bind to distinct, non-overlapping sites on the receptor binding domain of the SARS-CoV-2 spike protein. Binding to either of these sites blocks the virus's ability to bind to its human cellular receptor, ACE2. By blocking virus entry into human cells, 2196+2130 can prevent or treat illness due to SARS-CoV-2 infection, COVID-19.

Formulations for administering the 2196 and 2130 antibodies in two separate pharmaceutical formulations and together in a single formulation were developed.

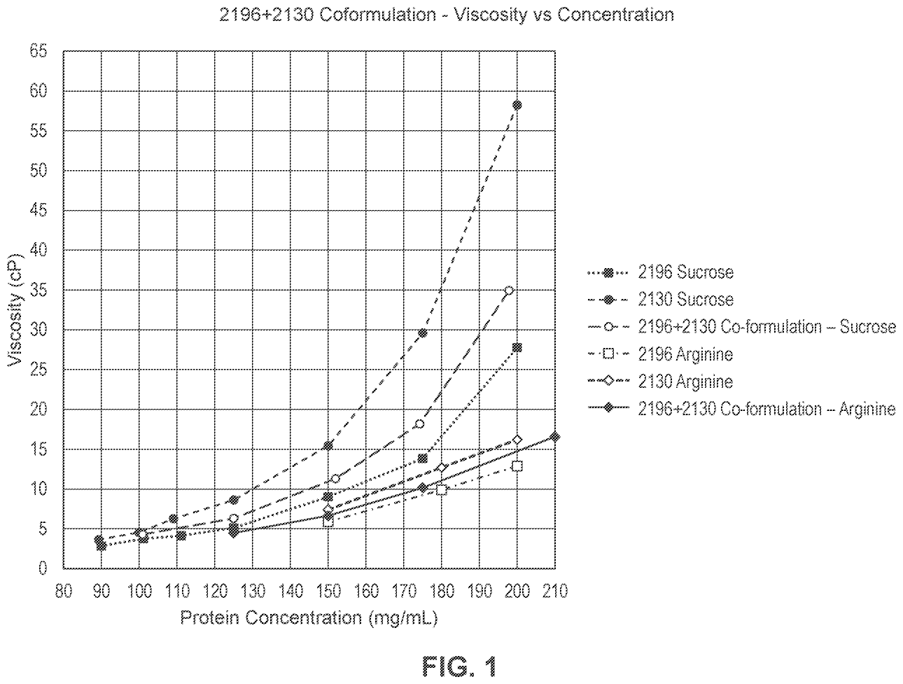

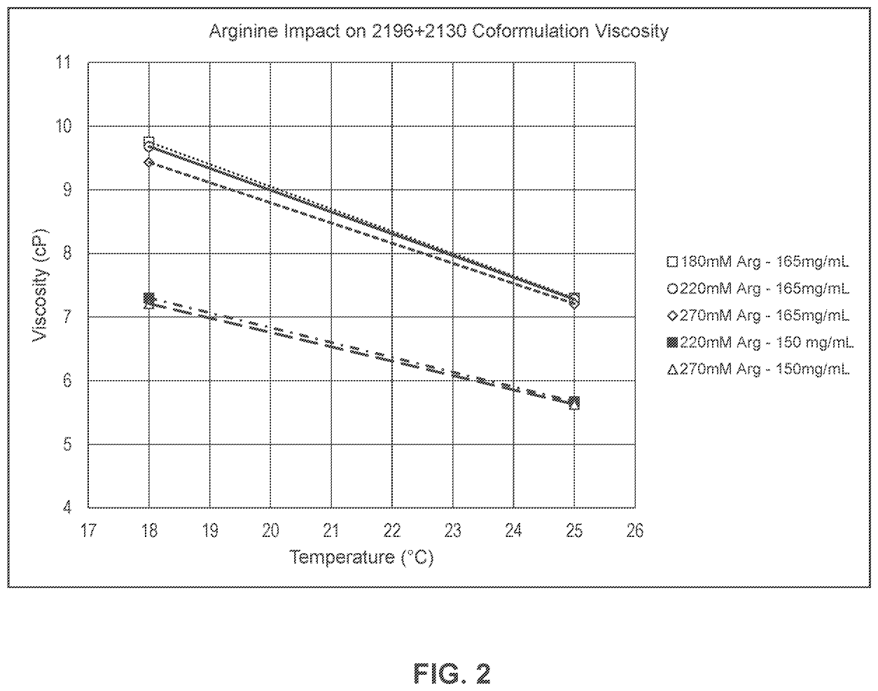

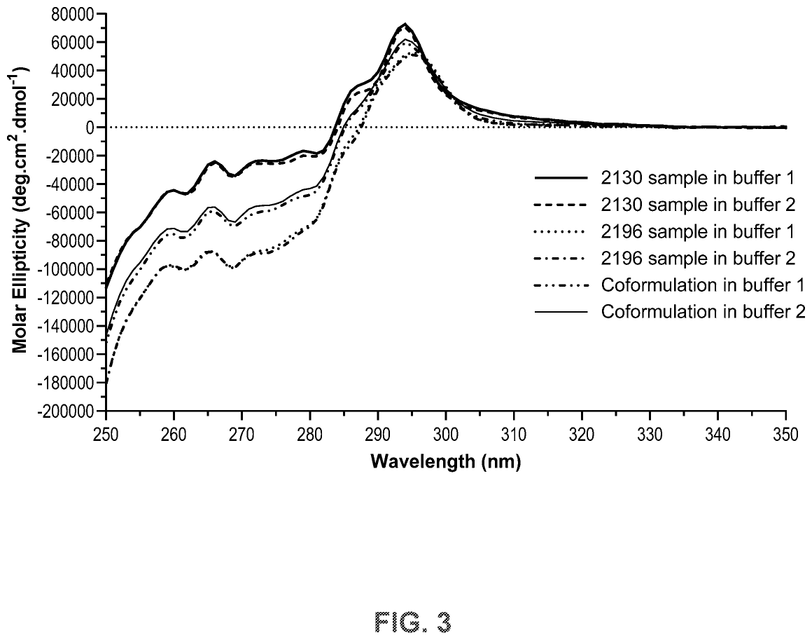

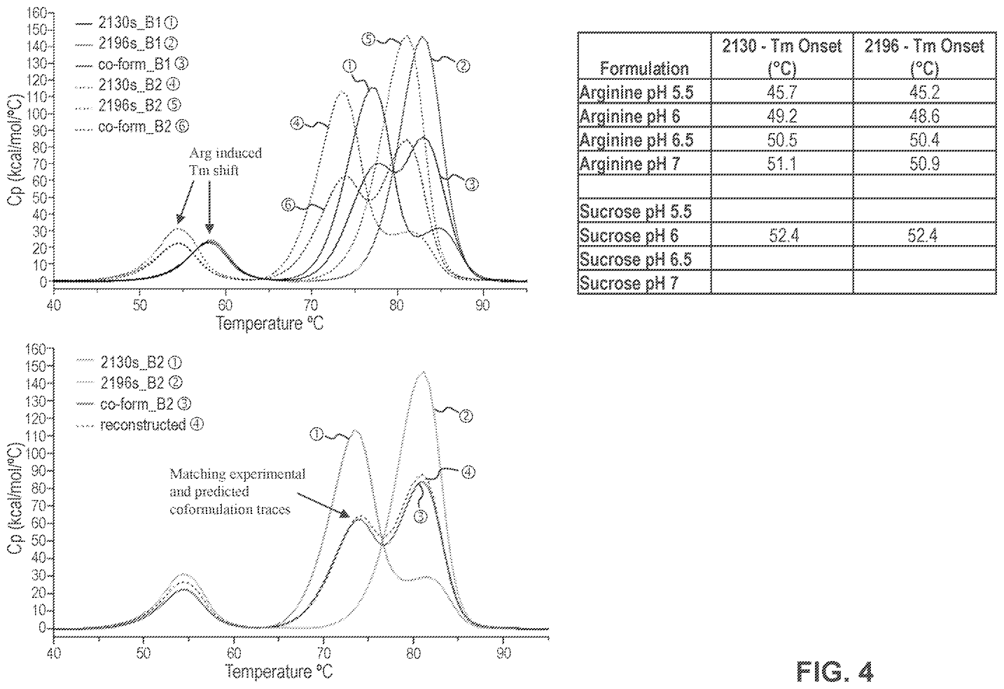

The viscosity of formulations tested is shown in FIG. 1. These data show that co-formulation mitigates the viscosity of 2130. The data in FIG. 2 demonstrate that viscosity is further mitigated by arginine, and arginine concentrations above 180 mM have minimal effect on 2196+2130 co-formulation viscosity. Additionally, no impact of pH was observed on co-formulation viscosity. In addition, near UV Circular Dichroism (CD (FIG. 3) analysis indicated that no changes to local structure resulted from co-formulation, and DSC data (FIG. 4) indicated that there were no changes to conformation stability due to co-formulation. A low CH2 domain melting temperature was driven by the YTE and TM mutations. A further shift in the melting temperature was driven by arginine (as compared to sucrose) and a lower pH. Each antibody exhibited favorable self-association characteristics as measured by DLS (kD>/˜20+ mL/gm).

Based on these assays, the following formulations were prepared for administration of the antibodies together in a single formulation (Treatment A) or in two separate formulations (Treatment B):

| Treatment Group | Treatment A | Treatment B |
|---|---|---|
| Formulation | 2196 + 2130 | 2196 and 2130 |
| | co-formulation of | in separate vials |
| | 2196 and 2130 in | |
| | the same vial | |
| | 20 mM histidine/ | 20 mM histidine/ |
| | histidine-HCl, | histidine-HCl, |
| | 220 mM | 240 mM sucrose, |
| | arginine-HCl, | 0.04% (w/v) |
| | 0.04% (w/v) | polysorbate |
| | polysorbate 80, | 80, pH 6.0 |
| | pH 6.0 | |
| Strength/ | 75 mg/mL 2196 + | 100 mg/mL 2196 |
| Concentration | 75 mg/mL | and 100 mg/mL |
| | 2130; | 2130 in separate |
| | ie, 150 mg/mL | vials |
| | total protein in a | |
| | single vial | |
| Dose (300 mg/) | 300 mg 2196 + | 150 mg 2196 and |
| | 2130 injection | 150 mg 2130 as |
| | (1 × 2 mL | two IM injections |
| | IM injection) | (1 × 1.5 mL IM |
| | | injection of each, |
| | | two injections total) |

### Example 2: Administration of Pharmaceutical Formulations Comprising Anti-SARS-CoV-2 Antibodies

A study is performed to compare 2196+2130 co-formulation (i) pharmacokinetic exposure and (ii) anti-Severe Acute Respiratory Syndrome Corona Virus 2 (SARS-CoV-2) neutralizing antibody levels in serum following intramuscular administration versus administration of 2196 and 2130 from two separate vials of the individual monoclonal antibodies in health adult participants.

The co-formulation and the administration of the separate vials is administered to healthy volunteers, male or female, ≥18 years of age, weighing≥50 to ≤110 kg, and with a body mass index of ≥18 to ≤30 kg/m². Participants are asked about anti-COVID-19 vaccination status. Participants are either non-vaccinated against SARS-CoV-2 infection or fully vaccinated with the last vaccine dose received at least 60 calendar days before the administration. Participants meet the following criteria:
Male and female participants, aged 18 years and older, fully vaccinated or non-vaccinated against SARS-CoV-2, with suitable veins for cannulation or repeated venipuncture. Vaccinated participants have received their last dose of the vaccine at least 60 calendar days before IMP administration (Day 1);
Participants have negative results of a SARS-CoV-2 RT-PCR test and non-vaccinated participants have negative results for a serology test within 2 weeks prior to randomization. Participants are either not vaccinated against SARS-CoV-2 infection or are fully vaccinated with the last vaccine dose received at least 60 calendar days before IMP dose administration (Day 1); and
Body weight≥50 kg to ≤110 kg at screening and a BMI≥18.0 to ≤30 kg/m² at the time of the Screening Visit

Patients receive injections at injection sites of lateral thigh (vastus lateralis), gluteal dorsal, or gluteal ventral. Participants receiving Treatment A are administered a dose via a single IM injection from a single drug product vial. Participants receiving Treatment B are administered a dose via two separate IM injections, each from a separate drug product vial containing 2196 or 2130. 2196 is administered first, followed by 2130.

Incidence and titers of 2196 and 2130 antibodies are evaluated to show that administration of either a co-formulation of the two antibodies or administration of the two antibodies in separate pharmaceutical formulations is effective in preventing and treating COVID-19

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1. A pharmaceutical formulation comprising:
(a) a first antibody or antigen-binding fragment thereof that binds to a spike protein of SARS-CoV-2 and optionally a second antibody or antigen-binding fragment thereof that binds to a spike protein of SARS-CoV-2,
(b) histidine and/or a pharmaceutically acceptable salt thereof,
(c) arginine and/or a pharmaceutically acceptable salt thereof or sucrose, and
(d) polysorbate,
wherein the formulation has a pH of about 5.5 to 6.5.

2. The pharmaceutical formulation of claim 1, wherein the formulation comprises about 15 mM to about 25 mM of (b), optionally wherein the formulation comprises about 20 mM of (b).

3. (canceled)

4. The pharmaceutical formulation of claim 1, wherein the formulation comprises about 200 to about 250 mM of (c).

5.-9. (canceled)

10. The pharmaceutical formulation of claim 1, wherein the formulation comprises about 0.03% to about 0.05% (w/v) of (d).

11.-12. (canceled)

13. A pharmaceutical formulation comprising:
(a) a first antibody or antigen-binding fragment thereof that binds to a spike protein of SARS-CoV-2 and a second antibody or antigen-binding fragment thereof that binds to a spike protein of SARS-CoV-2,
(b) about 20 mM histidine and/or a pharmaceutically acceptable salt thereof,
(c) about 220 mM arginine and/or a pharmaceutically acceptable salt thereof, and
(d) about 0.04% (w/v) polysorbate80,
wherein the formulation has a pH of about 6.0.

14. The pharmaceutical formulation of claim 13, wherein the formulation comprises about 135 mg/mL to about 165 mg/mL of (a).

15.-18. (canceled)

19. The pharmaceutical formulation of claim 1, wherein
the first antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and/or
the second antibody or antigen-binding fragment thereof comprises a VH CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

20. The pharmaceutical formulation of claim 1, wherein the first antibody or antigen-binding fragment thereof comprises a variable heavy chain (VH) comprising the amino acid sequence of SEQ ID NO:7 and a variable light chain (VL) comprising the amino acid sequence of SEQ ID NO:8; and/or the second antibody or antigen-binding fragment thereof comprises a variable heavy chain (VH) comprising the amino acid sequence of SEQ ID NO:15 and a variable light chain (VL) comprising the amino acid sequence of SEQ ID NO:16.

21.-22. (canceled)

23. The pharmaceutical formulation of claim 20, wherein the first antibody or antigen-binding fragment thereof comprises a YTE mutation and/or the second antibody or antigen-binding fragment thereof comprises a YTE mutation.

24. The pharmaceutical formulation of claim 20, wherein the first antibody or antigen-binding fragment thereof comprises a TM mutation and/or the second antibody or antigen-binding fragment thereof comprises a TM mutation.

25. The pharmaceutical formulation of claim 1, wherein the first antibody or antigen-binding fragment thereof comprises a heavy chain comprising amino acids 1-452 of SEQ ID NO:17 and a light chain comprising the amino acid sequence of SEQ ID NO:18 and/or wherein the second antibody or antigen-binding fragment thereof comprises a heavy chain comprising amino acids 1-460 of SEQ ID NO:19 and a light chain comprising the amino acid sequence of SEQ ID NO:20.

26. A pharmaceutical formulation comprising:
(a) an antibody or antigen-binding fragment thereof that binds to a spike protein of SARS-CoV-2,
(b) about 20 mM histidine/histidine-HCl,
(c) about 240 mM sucrose, and
(d) about 0.04% (w/v) polysorbate 80,
wherein the formulation has a pH of 6.0.

27.-29. (canceled)

30. The pharmaceutical formulation of claim 26, wherein
the antibody or antigen-binding fragment thereof comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; or
the antibody or antigen-binding fragment thereof comprises a CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14.

31. The pharmaceutical formulation of claim 26, wherein
the antibody or antigen-binding fragment thereof comprises a variable heavy chain (VH) comprising the amino acid sequence of SEQ ID NO:7 and a variable light chain (VL) comprising the amino acid sequence of SEQ ID NO:8; or
the antibody or antigen-binding fragment thereof comprises a variable heavy chain (VH) comprising the amino acid sequence of SEQ ID NO:15 and a variable light chain (VL) comprising the amino acid sequence of SEQ ID NO:16.

32.-33. (canceled)

34. The pharmaceutical formulation of claim 31, wherein the antibody or antigen-binding fragment thereof comprises a YTE mutation.

35. The pharmaceutical formulation of claim 31, wherein the antibody or antigen-binding fragment thereof comprises a TM mutation.

36. The pharmaceutical formulation of claim 26, wherein
the antibody or antigen-binding fragment thereof comprises a heavy chain comprising amino acids 1-452 of SEQ ID NO:17 and a light chain comprising the amino acid sequence of SEQ ID NO:18; or
the antibody or antigen-binding fragment thereof comprises a heavy chain comprising amino acids 1-460 of SEQ ID NO:19 and a light chain comprising the amino acid sequence of SEQ ID NO:20.

37.-40. (canceled)

41. A vial comprising the pharmaceutical formulation of claim 1.

42. (canceled)

43. A kit comprising a first pharmaceutical formulation of claim 26 and a second pharmaceutical formulation, wherein
the first formulation comprises an antibody or antigen-binding fragment thereof comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6; and
the second formulation comprises fa) an antibody or antigen-binding fragment thereof comprising a CDR1 comprising the amino acid sequence of SEQ ID NO:9, a VH CDR2 comprising the amino acid sequence of SEQ ID NO:10, a VH CDR3 comprising the amino acid sequence of SEQ ID NO:11, a VL CDR1 comprising the amino acid sequence of SEQ ID NO:12, a VL CDR2 comprising the amino acid sequence of SEQ ID NO:13, and a VL CDR3 comprising the amino acid sequence of SEQ ID NO:14 (b) histidine and/or a pharmaceutically acceptable salt thereof, (c) arginine and/or a pharmaceutically acceptable salt thereof or sucrose, and (d) polysorbate, wherein the second formulation has a pH of about 5.5 to 6.5.

44.-46. (canceled)

47. A method of treating or preventing Coronavirus Disease 2019 (COVID-19) in a subject comprising administering the pharmaceutical formulation of claim 1 to the subject.

48.-57. (canceled)


SEQUENCE LISTING (22 sequences)

SEQ ID NO: 1 - protein, 8 aa, synthetic construct
  source 1..8: note = CDR1
GFTFMSSA

SEQ ID NO: 2 - protein, 8 aa, synthetic construct
  source 1..8: note = CDR2
IVIGSGNT

SEQ ID NO: 3 - protein, 16 aa, synthetic construct
  source 1..16: note = CDR3
AAPYCSSISCNDGFDI

SEQ ID NO: 4 - protein, 7 aa, synthetic construct
  source 1..7: note = CDR1
QSVSSSY

SEQ ID NO: 5 - skipped

SEQ ID NO: 6 - protein, 10 aa, synthetic construct
  source 1..10: note = CDR3
QHYGSSRGWT

SEQ ID NO: 7 - protein, 123 aa, synthetic construct
  source 1..123: note = heavy chain variable region
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSS

SEQ ID NO: 8 - protein, 109 aa, synthetic construct
  source 1..109: note = light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIK

SEQ ID NO: 9 - protein, 8 aa, synthetic construct
  source 1..8: note = CDR1
GFTFRDVW

SEQ ID NO: 10 - protein, 10 aa, synthetic construct
  source 1..10: note = CDR2
IKSKIDGGTT

SEQ ID NO: 11 - protein, 22 aa, synthetic construct
  source 1..22: note = CDR3
TTAGSYYYDTVGPGLPEGKFDY

SEQ ID NO: 12 - protein, 12 aa, synthetic construct
  source 1..12: note = CDR1
QSVLYSSNNKNY

SEQ ID NO: 13 - skipped

SEQ ID NO: 14 - protein, 8 aa, synthetic construct
  source 1..8: note = CDR3
QQYYSTLT

SEQ ID NO: 15 - protein, 131 aa, synthetic construct
  source 1..131: note = Heavy chain variable region
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 16 - protein, 112 aa, synthetic construct
  source 1..112: note = light chain variable region
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 17 - protein, 453 aa, synthetic construct
  source 1..453: note = full length heavy chain
QMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 216 aa, synthetic construct
  source 1..216: note = full length light chain
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 461 aa, synthetic construct
  source 1..461: note = full length heavy chain
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, synthetic construct
  source 1..219: note = full length light chain
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 232 aa, synthetic construct
  source 1..232: note = IgG1
EPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 1273 aa, synthetic construct
  source 1..1273: note = spike protein of SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT